(19) United States
(12) Patent Application Publication
(10) Pub. No.: US 2010/0098735 A1
(43) Pub. Date: Apr. 22, 2010

(54) INJECTABLE DEPOT COMPOSITIONS AND ITS PROCESS OF PREPARATION

(76) Inventors: Rajesh Jain, New Delhi (IN); Kour Chand Jindal, New Delhi (IN); Sampath Kumar Devarajan, New Delhi (IN)

Correspondence Address: LADAS & PARRY LLP, 26 WEST 61ST STREET, NEW YORK, NY, 10023, US

(21) Appl. No.: 12/444,257

(22) PCT Filed: Oct. 3, 2007
(86) PCT No.: PCT/IN2007/000473

§ 371 Date: Apr. 3, 2009

(30) Foreign Application Priority Data
Oct. 5, 2006 (IN) 2195/DEL/2006

(51) Int. Cl. A61K 9/00; A61K 31/4196; A61K 31/18; A61K 9/14; A61P 35/00

(52) U.S. Cl. 424/422; 514/383; 514/603; 424/489

(57) ABSTRACT

Novel injectable compositions are provided comprising an active agent which is tamsulosin or letrozole or its pharmaceutically acceptable salts, derivatives, isomers, polymorphs, solvates, hydrates, analogues, enantiomers, tautomeric forms or mixtures thereof and one or more pharmaceutically acceptable excipient(s) wherein the compositions are preferably formulated as biodegradable microparticles or nanoparticles which can optionally be reconstituted with an aqueous, hydro-alcoholic or oily liquid vehicle prior to administration. The novel injectable compositions of the present invention preferably form a depot upon administration in vivo and are in the form of an in situ gelling composition or an implant composition which provides a prolonged release of tamsulosin or letrozole for extended periods of time. Also described are process for preparation of such novel compositions and method of using them.

### FIELD OF THE INVENTION

The present invention relates to novel injectable compositions comprising an active agent which is tamsulosin or letrozole or its pharmaceutically acceptable salts, derivatives, isomers, polymorphs, solvates, hydrates, analogues, enantiomers, tautomeric forms or mixtures thereof and one or more pharmaceutically acceptable excipient(s) wherein the compositions are preferably formulated as biodegradable microparticles or nanoparticles which can optionally be reconstituted with an aqueous, hydro-alcoholic or oily liquid vehicle prior to administration. The novel injectable compositions of the present invention preferably form a depot upon administration in vivo and are in the form of an in situ gelling composition or a monolithic implant composition which provides a prolonged release of tamsulosin or letrozole for extended periods of time. The present invention also describes process for preparation of such novel compositions and method of using such compositions.

### BACKGROUND OF THE INVENTION

Aromatase inhibitors are a class of compounds that act systematically to inhibit oestrogen synthesis in tissues. These compounds prevent oestrogen biosynthesis by inhibiting the enzyme aromatase, which catalyses the conversion of adrenal androgens (androstenedione and testosterone) to oestrogens (oestrogen and oestradiol). There has therefore been interest in developing these compounds as potential therapies for hormone responsive breast cancer in post-menopausal women. Letrozole is a nonsteroidal competitive inhibitor of the aromatase enzyme system; it inhibits the conversion of androgens to estrogens. Letrozole selectively inhibits gonadal steroidogenesis but has no significant effect on adrenal mineralocorticoid or glucocorticoid synthesis. Treatment of women with letrozole significantly lowers serum estrone, estradiol and estrone sulfate. Letrozole is commercially available under the trade name FEMARA® as 2.5 mg tablets for oral administration. Tamsulosin is an alpha 1-adrenoceptor blocking agent, which exhibits selectivity for alpha 1-receptors in the human prostate. Tamsulosin is used for the treatment of the symptoms associated with benign prostatic hyperplasia, such as bladder outlet obstruction, which is comprised of static and dynamic components. Tamsulosin is commercially available as its hydrochloride salt under the trade name FLOMAX™ as 0.4 mg oral capsule.

It is often desirable to administer drugs using controlled or sustained release formulations that can maintain therapeutic blood levels of the active agent (drug) over extended periods of time. These controlled release formulations reduce the frequency of dosing for enhanced patient convenience and compliance, and also reduce the severity and frequency of side effects. By maintaining substantially constant blood levels and avoiding blood level fluctuations of the drug particularly associated with conventional immediate release formulations that are administered several times a day, controlled or sustained release formulations can provide a better therapeutic profile than is obtainable with conventional immediate release formulations. It is also often desirable to extend the release time of an injected drug to increase its duration of action, or to reduce its toxic effects. Formulations that are readily soluble in the body are usually absorbed rapidly and provide a sudden burst of available drug as opposed to a more desirable and gradual release of the pharmacologically active agent. This ‘burst’ release often results in a substantial portion of the beneficial agent, if not all, being released in a very short time, e.g., hours or 1-2 days. Several attempts have been made to provide controlled release compositions, but have not succeeded in overcoming certain problems associated with long acting parenteral dosage forms, such as achieving an extended release over desired period, stability in tissue fluids, reduced toxicity, reproducibility in preparation, and the elimination of undesired physical, biochemical, or toxicological effects associated with the compositions.

Where patient compliance is an issue, a probable approach is to design long acting dosage form compositions of the medication, that is, dosage forms where a single administration leads to a sustained release of the medication over an extended period of time. This, in turn, simplifies the dosage regimen that a patient needs to adhere to, thus reducing the opportunity for non-compliance that occurs with a more rigorous schedule of frequent administration. Among such dosage forms is the depot formulation, which can be administered in various ways including intramuscularly or subcutaneously by injection. The depot injection is specifically formulated to provide a sustained release of the medication, over an extended period of time like days, weeks, months or even up to years, as in case of parenteral sustained release formulations.

The use of injectable implants for the delivery of drugs is well known. Both biodegradeable and non-biodegradeable implant versions have been marketed since the 1980s. Examples of these are Zoladex®, a polylactide-co-glycolide formulation of goserelin for the treatment of breast cancer and Norplant®, a non-biodegradeable silicone device for contraception. Small, injectable microparticle formulations are also well known, an example being Lupron Depot®, a formulation of leuprolide for the treatment of prostate cancer. A drawback of such preformed delivery systems is administration. Cylindrical rods such as Zoladex® require relatively large bore needles for implantation. However, injectable formulations comprising microparticles or nanoparticles allow smaller bore needles to be used for in vivo administration. More recently formulations have been developed which are injected as a liquid, but undergo a change to a solid formulation in vivo, which are referred to as ‘in situ gelling systems’. These formulations can be injected intramuscularly or subcutaneously through small bore needles and employ only biocompatible solvents.

US Publication No. 20020034532 discloses injectable depot gel composition comprising a biocompatible polymer; a solvent that dissolves the biocompatible polymer and forms a viscous gel; a beneficial agent; and an emulsifying agent in the form of a dispersed droplet phase in the viscous gel. U.S. Pat. No. 6,287,588 claims a dual phase polymeric agent-delivery composition comprising a continuous biodegradable hydrogel phase, a discontinuous particulate phase comprising defined microparticles; and an agent to be delivered contained in at least said discontinuous particulate phase. The bioactive agent release is described to be modulated by microparticle phase alone or in both the microparticle and the gel matrix. The invention describes a reverse thermal gelation type of matrix. However, the said invention does not describe through clear illustrations the polymeric hydrogel formation at the injection site by non solvent effect or by using an unhydrated cellulosic polymer in the reconstituted suspension composition having easy syringibility to be used as a depot injection.

German patent no. DE19847593 relates to a composition for parenteral administration comprising an active agent and a carrier material consisting of spherical microparticles of average diameter 1 nm to 100 μm, and at least partly of water-insoluble linear polysaccharide. US publication no. 20050153841 discloses a formulation for parenteral administration to a subject, comprising at least one water miscible solvent; at least one gelling agent; and at least one active agent; characterized in that the gelling agent is in particulate form and suspended in the solvent. However, the said invention does not describe the dual modulation of drug release patterns by means of simultaneously using gelling system dispersed with release controlling particulate form of drug in biodegradable microparticles. PCT Publication No. WO 2006/099121 describes a pharmaceutical composition comprising particles of dutasteride, tamsulosin hydrochloride, or a combination thereof having an effective average particle size of less than about 2000 nm; and at least one surface stabilizer. However, no specific injectable composition comprising tamsulosin as microparticles had been disclosed.

No specific injectable depot compositions comprising tamsulosin or letrozole particularly formulated as microparticles had been disclosed in the prior art; instead only solid oral immediate release compositions are being marketed comprising the said drugs. Hence, there is an unmet need for compositions comprising tamsulosin or letrozole for long-term use that is clinically tolerable, effective and safe, has a low potential for morbidity, and is cost-effective. Such compositions would highly improve patient compliance since they would abolish the need for daily administration of the drug for substantially long duration, of treatment. However, in general, several attempts to provide dosage form compositions to sustain medication levels including the use of biodegradable materials for delivery of active agent for extended periods of time have been described previously. Many sustained release parenteral compositions described in the prior art can exhibit an increased release of biologically active agent over the first twenty-four hours after administration, commonly referred to as a ‘burst’. In some instances, this burst can result in an undesirable increase in the levels of biologically active agent leading to toxic effects and/or minimal release of agent thereafter providing sub-therapeutic concentration of active agent (drug). Therefore, a need still exists for providing sustained release parenteral depot compositions of tamsulosin and letrozole where a proper control over release kinetics by, for example, reducing the burst release of drug can be exerted and a continuous release of drug for longer period of duration, for example, for a week or a month or 3 months or more can be achieved, yet possessing good syringibility' characteristics.

### SUMMARY OF THE INVENTION

It is an objective of the present invention to provide novel injectable compositions comprising an active agent which is tamsulosin or letrozole or its pharmaceutically acceptable salts, derivatives, isomers, polymorphs, solvates, hydrates, analogues, enantiomers, tautomeric forms or mixtures thereof, at least one biodegradable polymer(s) and optionally one or more pharmaceutically acceptable excipient(s), wherein the compositions are formulated as biodegradable microparticles or nanoparticles, and wherein the said compositions provide a prolonged release of tamsulosin or letrozole for extended periods of time.

It is an objective of the present invention to provide novel injectable compositions comprising an active agent which is tamsulosin or letrozole or its pharmaceutically acceptable salts, derivatives, isomers, polymorphs, solvates, hydrates, analogues, enantiomers, tautomeric forms or mixtures thereof, at least one biodegradable polymer(s) and optionally one or more pharmaceutically acceptable excipient(s), wherein the compositions are formulated as biodegradable microparticles or nanoparticles which can be reconstituted with an aqueous, hydro-alcoholic or oily liquid vehicle prior to administration.

It is an objective of the present invention to provide novel injectable compositions comprising an active agent which is tamsulosin or letrozole or its pharmaceutically acceptable salts, derivatives, isomers, polymorphs, solvates, hydrates, analogues, enantiomers, tautomeric forms or mixtures thereof, at least one biodegradable polymer(s) and optionally one or more pharmaceutically acceptable excipient(s), which are in the form of an in situ gelling composition or a implant composition and which form a depot upon administration in vivo upon contact with body fluids therefore providing a prolonged release of the active agent for extended periods of time.

It is an objective of the present invention to provide novel injectable compositions comprising tamsulosin or letrozole as active agent in an amount of from about 0.1% w/w to about 95% w/w, at least one biodegradable polymer(s) in an amount of from about 0.1% w/w to about 95% w/w, optionally one or more pharmaceutically acceptable excipient(s) in an amount of from about 0.1% to about 99.8% w/w based upon the total weight of the formulation, wherein the biodegradable polymer(s) is a polylactide polymer or a polyglycolide polymer or a poly(lactide-co-glycolide) co-polymer having an average molecular weight of from about 1,000 Daltons to about 200,000 Daltons; and wherein the said composition forms a gel or implant when placed in an aqueous physiological-type environment and releases the active agent for over a period of at least 7 days.

It is an objective of the present invention to provide novel injectable compositions comprising an active agent which is tamsulosin or letrozole or its pharmaceutically acceptable salts, derivatives, isomers, polymorphs, solvates, hydrates, analogues, enantiomers, tautomeric forms or mixtures thereof, at least one biodegradable polymer(s) and optionally one or more pharmaceutically acceptable excipient(s), wherein the compositions are formulated as biodegradable microparticles or nanoparticles, and wherein the said compositions are in the form of a multi-component system preferably comprising at least two components.

It is an objective of the present invention to provide novel injectable depot compositions of tamsulosin or letrozole comprising of at least two components, wherein component-1 is in the form of a readily dispersible composition preferably as microparticles or nanoparticles comprising tamsulosin or letrozole and at least one biodegradable polymer(s), optionally with one or more pharmaceutical acceptable excipient(s); and wherein component-2 is in the form of a liquid vehicle for reconstitution of component-1 comprising at least one water miscible or water immiscible solvent, optionally with one or more pharmaceutical acceptable excipient(s); and wherein the compositions comprise at least one viscosity enhancing agent(s) either present in component-1 or component-2 or both. The viscosity enhancing agent(s) is either present in component-1 or component-2 or both in an unhydrated form.

It is an objective of the present invention to provide novel injectable depot compositions of tamsulosin or letrozole comprising of at least two components, wherein component-1 is in the form of biodegradable microparticles or nanoparticles comprising tamsulosin or letrozole as active agent, at least one biodegradable polymer(s), at least one viscosity enhancing agent(s) and optionally one or more pharmaceutical acceptable excipient(s); wherein the biodegradable microparticle or nanoparticle is partially or entirely embedded in the viscosity enhancing agent which acts as release modifier upon contact with body fluids by getting hydrated and forming a gel around the biodegradable microparticles.

It is an objective of the present invention to provide novel injectable depot compositions of tamsulosin or letrozole comprising of at least two components, wherein component-1 is in the form of biodegradable microparticles or nanoparticles comprising tamsulosin or letrozole as active agent, at least one biodegradable polymer(s), at least one viscosity enhancing agent(s) and optionally one or more pharmaceutically acceptable excipients; wherein the viscosity enhancing agent(s) is a biocompatible cellulosic polymer which acts as active agent release modifier and/or a gel forming agent.

It is also an objective of the present invention to provide novel injectable depot compositions of tamsulosin or letrozole which provides a flowable composition for forming a solid or semi-solid biodegradable gel or implant in situ within a body, comprising at least one biodegradable polymer(s), at least one viscosity enhancing agent(s) and optionally at least one biocompatible solvent(s) that at least partially solubilizes the biodegradable polymer(s) and/or the viscosity enhancing agent(s) and is miscible or dispersible in aqueous body fluids, and capable of dissipating, diffusing or leaching from the composition into body fluid upon placement within a body, whereupon the biodegradable polymer(s) and/or the viscosity enhancing agent(s) coagulate or precipitate to form the gel or implant.

It is another objective of the present invention to provide process for preparation of such novel injectable compositions which comprises preparation of tamsulosin or letrozole microparticles or nanoparticles and optionally a liquid vehicle in which the said microparticles or nanoparticles may be reconstituted prior to administration.

It is another objective of the present invention to provide a method of forming a depot gel or an implant in situ, in a living body, which comprises preparing an in situ gelling formulation according to the method described herein, placing the formulation within the body and allowing the liquid vehicle to disperse or dissipate to produce a solid or gel implant.

It is yet another objective of present invention to provide a pharmaceutical kit suitable for in situ formation of a biodegradable depot gel or implant from the novel compositions as described herein, in the body of a subject in need thereof, which comprises a device containing tamsulosin or letrozole microparticles and optionally one or more pharmaceutical acceptable excipient(s), and a device containing liquid vehicle and optionally one or more pharmaceutical acceptable excipient(s); wherein the devices allow for expulsion of contents of the two devices for enabling mixing together prior to administration of contents into the body of subject.

It is still another objective of the present invention to provide use of an in situ gelling formulation as described herein in the manufacture of a medicament for the treatment of a condition treatable by tamsulosin or letrozole in a Mammal particularly a human being.

It is yet another objective of the present invention to provide a method of using the compositions of tamsulosin or letrozole according to the present invention which comprises administering to a subject/patient in need thereof an effective amount of the said composition. The novel compositions of the present invention comprising tamsulosin is particularly useful for management such as prophylaxis, amelioration and/or treatment of subjects for the signs and symptoms of benign prostatic hyperplasia and compositions of the present invention comprising letrozole is particularly useful for management such as prophylaxis, amelioration and/or treatment of hormonally-responsive breast cancer.

### DETAILED DESCRIPTION OF THE INVENTION

The present invention provides novel injectable compositions comprising an active agent which is tamsulosin or letrozole, at least one biodegradable polymer(s) and optionally one or more pharmaceutically acceptable excipient(s), wherein the compositions are formulated as biodegradable microparticles or nanoparticles, and wherein the said compositions provide a prolonged release of tamsulosin or letrozole for extended periods of time. The active agent ‘tamsulosin or letrozole’ wherever disclosed in the entire description hereinafter also encompasses its pharmaceutically acceptable salts, polymorphs, solvates, hydrates, analogues, enantiomers, tautomeric forms, derivatives or mixtures thereof unless otherwise mentioned.

Both the drugs tamsulosin and letrozole are presently available as oral dosage forms for daily administration. It is mandatory for the patients in need thereof to take the drugs daily to achieve desired therapeutic plasma concentrations for optimum therapeutic benefit. Patient compliance with such a daily dosing regimen is however, difficult to ensure, especially where the course of therapy is long or of intermediate or lifetime duration. Thus, there is a need for a prolonged release formulation of tamsulosin and letrozole to improve patient compliance/convenience and give patients optimum therapeutic benefit by abolishing the need to administer a dosage composition daily, which the present invention provides in the form of injectable compositions. The novel injectable compositions of the present invention leads to less frequent dosing of drugs, and still provides an improved therapeutic effect with reduced side effects by effectively smoothening out the fluctuations in the plasma concentration-time profile. Most importantly, the prolonged release formulations of the present invention improves the ‘quality of life’ of patients undertaking treatment with letrozole for oncology indications and with tamsulosin for Benign Prostatic Hyperplasia (BPH) which is particularly seen in older individuals.

The novel injectable composition of the present invention comprising effective dose of tamsulosin or letrozole are required to be administered in substantially low volumes which are convenient to administer and causes minimal pain on injection. Further the in situ gelling depot or implant compositions of the present invention are designed in such a manner so as to exhibit a gradual partitioning out of the depot during the depot formation stage upon in vivo administration thus leading to surprisingly low initial ‘burst’ release of the active agent. This in turn alleviates possibility of any side effects and enhances the ‘life’ of the depot in producing sustained release of the active agent for extended time duration.

In an embodiment, the present invention provides novel injectable compositions comprising an active agent which is tamsulosin or letrozole, at least one biodegradable polymer(s) and optionally one or more pharmaceutically acceptable excipient(s), wherein the compositions are formulated as biodegradable microparticles or nanoparticles which can be reconstituted with an aqueous, hydro-alcoholic or oily liquid vehicle prior to administration. The novel compositions are in the form of an in situ gelling composition or a monolithic implant composition which form a depot upon administration in vivo upon contact with body fluids therefore providing a prolonged release of the active agent for extended periods of time. The novel compositions of the present invention are capable of producing a prolonged release of tamsulosin or letrozole for at least 7 days preferably for a period of at least 15 days to 6 months, or more.

In another embodiment, the present invention provides novel injectable in situ gelling depot or implant compositions exhibiting minimal burst release of the active agent which is achieved by the formation of a substantially cohesive gel-like mass due to gradual swelling of viscosity enhancing agent(s) in the aqueous physiological-type environment sufficient to form a solid or semisolid depot gel or implant shortly after the composition is administered into a living host. The compositions of the present invention comprises microparticles or nanoparticles of the active agent which gets embedded in the in situ gelled matrix formed upon in vivo administration; hence providing a dual mechanism for controlling the drug release i.e. the controlled release provided by the biodegradable polymer(s) and the gelled matrix formed due to the gelling of the viscosity enhancing polymer(s) upon contact with body fluids.

The novel injectable compositions of the present invention leads to less frequent dosing of drugs, and still provides an improved therapeutic effect with reduced side effects by effectively smoothening out the fluctuations in the plasma concentration-time profile. Most importantly, the prolonged release formulations of the present invention improves the ‘quality of life’ of patients undertaking long term treatment for chronic diseases/disorders such as cancers, psychosis, and the like. The novel injectable composition of the present invention comprising effective dose of tamsulosin or letrozole is required to be administered in substantially low volumes which are convenient to administer and causes minimal pain on injection. Further the in situ gelling depot or implant compositions of the present invention are designed in such a manner so as to exhibit a gradual partitioning out of the depot during the depot formation stage upon in vivo administration thus leading to surprisingly low initial ‘burst’ release of the active agent. This in turn alleviates possibility of any side effects and enhances the ‘life’ of the depot in producing sustained release of the active agent for extended time duration.

The novel depot injectable compositions of the present invention are able to provide a sustained release of the active agent for a prolonged duration even by using substantially low quantities of high molecular weight hydrophobic polymers such as the polylactide polymer or a polyglycolide polymer or a poly(lactide-co-glycolide) co-polymer thus resulting in less residual polymer remaining at the site of administration after the release of active core. Further, the compositions of present invention are formulated such that the chances of dose dumping due to failure of system is avoided or substantially reduced upon in vivo administration to a subject.

In an embodiment of the present invention is provided novel injectable compositions comprising tamsulosin or letrozole as active agent in an amount of from about 0.1% w/w to about 95% w/w, at least one biodegradable polymer(s) in an amount of from about 0.1% w/w to about 95% w/w, optionally one or more pharmaceutically acceptable excipient(s) in an amount of from about 0.1% to about 99.8% w/w based upon the total weight of the formulation, wherein the biodegradable polymer(s) is a polylactide polymer (PLA) or a polyglycolide polymer or a poly(lactide-co-glycolide) co-polymer (PLGA) having an average molecular weight of from about 1,000 Daltons to about 200,000 Daltons; and wherein the said composition forms a gel or implant when placed in an aqueous physiological-type environment and releases the active agent for over a period of at least 7 days. In an embodiment of the present invention is provided novel injectable compositions comprising tamsulosin or letrozole as active agent and at least one biodegradable polymer(s), wherein the ratio of active agent to the biodegradable polymer(s) is between about 1:100 to about 100:1.

The present invention provides novel injectable depot compositions of tamsulosin or letrozole which are flowable and which are capable of forming a solid or semi-solid biodegradable gel or implant in situ within a body. In another embodiment, the present invention provides an in situ gelling composition comprising the active agent and a biodegradable polymer, dissolved dispersed or suspended in suitable liquid vehicle such as an aqueous vehicle or an oily vehicle. The compositions of the invention, upon contact with water or bodily fluids, result in the precipitation of both the polymer and the active agent (tamsulosin or letrozole) and subsequent formation of a gel or an implant within which the active agent is incorporated. Tamsulosin or letrozole subsequently diffuses from the gel or implant over an extended period of time to provide the desired pharmacological effect. In still other embodiments, the active agent may be encapsulated or otherwise incorporated into particles, such as microspheres, nanospheres, liposomes, lipospheres, micelles, and the like, or it may be conjugated to a polymeric carrier.

In another embodiment, the microparticles or nanoparticles of tamsulosin or letrozole useful for formulating the injectable composition are produced by a method which comprises spray-drying a solution or suspension comprising tamsulosin or letrozole. In yet another embodiment, the injectable composition of the present invention comprising microparticles or nanoparticles of tamsulosin or letrozole can be delivered through a parenteral, transdermal, transmucosal or subcutaneous route using a needleless syringe. In an embodiment, the present invention provides a single component system for administration of tamsulosin or letrozole by injection. In such case, the final product comprising tamsulosin or letrozole is present as a powder optionally mixed with a pharmaceutically acceptable gelling agent and/or a surfactant that can be administered directly into the subject's body.

In another embodiment, the present invention provides novel injectable compositions comprising an active agent which is tamsulosin or letrozole, at least one biodegradable polymer(s) and optionally one or more pharmaceutically acceptable excipient(s), wherein the compositions are formulated as biodegradable microparticles or nanoparticles, and wherein the said compositions are in the form of a multi-component system preferably comprising at least two components namely component-1 and component-2. In accordance with as aspect of the present invention is provided novel injectable depot compositions of tamsulosin or letrozole comprising of at least two components, wherein component-1 is in the form of a readily dispersible composition preferably as microparticles or nanoparticles comprising tamsulosin or letrozole and at least one biodegradable polymer(s), optionally with one or more pharmaceutical acceptable excipient(s); and wherein component-2 is in the form of a liquid vehicle for reconstitution of component-1 comprising at least one water miscible or water immiscible solvent, optionally with one or more pharmaceutical acceptable excipient(s); and wherein the compositions comprise at least one viscosity enhancing agent(s) either present in component-1 or component-2 or both. The viscosity enhancing agent(s) is either present in component-1 or component-2 or both in an unhydrated form.

In another embodiment, the present invention provides injectable depot compositions of tamsulosin or letrozole comprising of at least two components, wherein component-1 is in the form of biodegradable microparticles or nanoparticles comprising tamsulosin or letrozole as active agent, at least one biodegradable polymer(s), at least one viscosity enhancing agent(s) and optionally one or more pharmaceutical acceptable excipient(s); wherein the biodegradable microparticles or nanoparticles are partially or entirely embedded in the viscosity enhancing agent which acts as release modifier upon contact with body fluids by getting hydrated and forming a gel around the biodegradable microparticles. In an aspect, the viscosity enhancing agent(s) is a biocompatible cellulosic polymer which acts as microparticle or nanoparticle stabilizer, active agent release modifier and/or a gel forming agent.

In an embodiment, the novel injectable depot compositions comprise of at least two component system, wherein component-1 comprises a readily dispersible composition preferably in the form of microparticles or nanoparticles which comprise tamsulosin or letrozole and at least one biodegradable polymer(s) optionally with channel forming agent(s) to form biodegradable microparticles or nanoparticles having desired drug release characteristics; and wherein component-2 is a liquid vehicle for reconstituting the component-1; and wherein the compositions comprise at least one viscosity enhancing agent(s) either present in component-1 or component-2 or both; and wherein the composition forms an in situ gel preferably at the site of injection upon contact with body fluids.

In an embodiment of the present invention, the biodegradable polymer is selected from but not limited to a group comprising lactic acid-based polymers such as polylactides e.g. poly (D,L-lactide) i.e. PLA; glycolic acid-based polymers such as polyglycolides (PGA) e.g. Lactel® from Durect; poly (D,L-lactide-co-glycolide) i.e. PLGA, (Resomer® RG-504, Resomer® RG-502, Resomer® RG-504H, Resomer® RG-502H, Resomer® RG-504S, Resomer® RG-502S, from Boehringer, Lactel® from Durect); polycaprolactones such as poly(e-caprolactone) i.e. PCL (Lactel® from Durect); polyanhydrides; poly(sebacic acid) SA; poly(ricenolic acid) RA; poly(fumaric acid), FA; poly(fatty acid dimmer), FAD; poly(terephthalic acid), TA; poly(isophthalic acid), IPA; poly(p-{carboxyphenoxy}methane), CPM; poly(p-{carboxyphenoxy}propane), CPP; poly(p-{carboxyphenoxy} hexane), CPH; polyamines, polyurethanes, polyesteramides, polyorthoesters {CHDM: Cis/trans-cyclohexyl dimethanol, HD:1,6-hexanediol. DETOU: (3,9-diethylidene-2,4,8,10-tetraoxaspiro undecane)}; polydioxanones; polyhydroxybutyrates; polyalkyene oxalates; polyamides; polyesteramides; polyurethanes; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic celluloses; polyacrylic acids; polybutyric acid; polyvaleric acid; polyethylene glycol; polyhydroxycellulose; chitin; chitosan; polyorthoesters and copolymers, terpolymers; dimethyl isosorbide; lipids such as cholesterol, lecithin; poly(glutamic acid-co-ethyl glutamate), and the like, or mixtures thereof.

Preferably the biodegradable polymer is a lactic acid-based polymer, more preferably polylactide or poly (D, L-lactide-co-glycolide) i.e. PLGA. Preferably the biodegradable polymer is present in an amount between about 5% to about 98% w/w of the component-1. The lactic acid-based polymer has a monomer ratio of lactic acid to glycolic acid in the range of 100:0 to about 0:100 preferably 100:0 to about 10:90 and has an average molecular weight of from about 1,000 to 200,000 daltons. It might be emphasized that the choice and the quantity of biodegradable polymer is governed by the nature and quantity of active agent used, the desired particle size of the composition, the intended use and the duration of use, and the like.

In another embodiment, the component-1 of the present invention additionally comprises excipients selected from but not limited to a group comprising channel forming agents, oily components, emulsifiers, preservatives, antioxidants, stabilizers or mixtures thereof.

In another embodiment of the present invention, a process of preparation of microparticles or nanoparticles involves preferably o/w emulsion technique followed by solvent evaporation. The microparticles or nanoparticles comprise an oil phase wherein the oil phase is selected from but not limited to a class of water immiscible solvents preferably having low boiling point such as esters (e.g. ethyl acetate, butyl acetate), halogenated hydrocarbons (e.g. dichloromethane, chloroform, carbon tetrachloride, chloroethane, dichloroethane, trichloroethane), ethers (e.g. ethyl ether, isopropyl ether), aromatic hydrocarbons (e.g. benzene, toluene, xylem), carbonates (e.g. diethyl carbonate), or the like or mixtures thereof. The oil phase also may comprise a mixture of water miscible solvent (e.g. Acetone) and water immiscible solvent (e.g. dichloromethane) in various proportions. Suitable emulsifiers are used in the preparation of the microparticles or nanoparticles to enhance the stabilization of oil droplets against coalescence, wherein the emulsifier is selected from but not limited to a group comprising polyoxyethylene sorbitan fatty acid esters e.g. mono- and tri-lauryl, palmityl, stearyl and oleyl esters; sorbitan fatty acid esters (SPAN®); polysorbates (Tween®), polyvinyl alcohol, polyvinyl pyrrolidone, gelatin, lecithin, polyoxyethylene castor oil derivatives (Cremophor®), particularly suitable are polyoxyl 35 castor oil (Cremophor®EL) and polyoxyl 40 hydrogenated castor oil (Cremophor®RH40); tocopherol; tocopheryl polyethylene glycol succinate (vitamin E TPGS); tocopherol palmitate and tocopherol acetate; polyoxyethylene-polyoxypropylene co-polymers (Pluronic® or Poloxamer®), Sodium CMC and the like or mixtures thereof. Suitable channel forming agents optionally used to formulate the microparticles or nanoparticles is selected from but not limited to a group comprising polyglycols, ethyl vinyl alcohols, glycerin, pentaerythritol, polyvinyl alcohols, polyvinyl pyrrolidone, vinyl pyrrolidone, N-methyl pyrrolidone, polysaccharides such as dextrines and/or hydrolyzed starch, saccharides, sugar alcohols and the like or mixtures thereof.

In an embodiment of present invention, the viscosity enhancing agent of component-1 is selected from but not limited to group comprising cellulose derivatives, such as hydroxypropyl cellulose, hydroxyethyl cellulose, hydroxypropyl methyl cellulose, methylcellulose, sodium carboxymethyl cellulose and its derivatives, vinyl polymers, polyoxyethylene-polyoxypropylene polymers or co-polymers (Pluronics®), polysaccharides such as glycosaminoglycans, agar, pectin, alginic acid, dextran, starch and chitosan; proteins, poly(ethyleneoxide), acrylamide polymers, polyhydroxy acids, polyanhydrides, polyorthoesters, polyamides, polycarbonates, polyalkylenes, polyalkylene glycols, polyalkylene oxides, polyalkylene terepthalates, polyvinyl alcohols, polyacrylic acid, polymethacrylic acid, polyvinyl pyrrolidone, polyvinyl ethers, polyvinyl esters, polyvinyl halides, polysiloxanes, polyvinyl acetates, polystyrene, polyurethanes, synthetic celluloses, polyacrylic acids, polybutyric acid, polyvaleric acid, poly(lactide-co-caprolactone), and copolymers, derivatives; or mixtures thereof. Preferably the viscosity enhancing agent(s) is a high viscosity grade of sodium carboxymethyl cellulose or methyl cellulose. Preferably viscosity enhancing agent is present in an amount between about 0.1% to about 50%, more preferably between about 0.5% to about 50% by weight of composition either in component-1 or component-2 or both.

In another embodiment of the present invention, the liquid vehicle (of component-2) is in the form of an aqueous vehicle comprising water and optionally water miscible solvent selected from but not limited to group comprising preferably a water-miscible alcohol, for example, ethanol, n-propyl alcohol, isopropyl alcohol, tert-butyl alcohol, or propylene glycol; dimethylsulfoxide; dimethylformamide; a water-miscible ether, for example tetrahydrofuran; a water-miscible nitrite, for example acetonitrile; a water-miscible ketone, for example acetone or methyl ethyl ketone; an amide, for example dimethylacetamide; glycerin; polyethylene glycol 400; glycofurol (tetraglycol), and the like; or mixtures thereof. Preferably the water miscible solvent useful in the present invention is selected from glycerin, ethanol, propylene glycol, polyethylene glycols, or mixtures thereof.

In another embodiment of the present invention, the liquid vehicle of the present invention is an oily vehicle comprising at least one oily component selected from but not limited to a group comprising vegetable oils such as corn oil, almond oil, sunflower oil, castor oil, and the like, or a lipophilic compound such as dimethyl isosorbide; optionally with a surfactant selected from a group comprising anionic, cationic, non-ionic or zwitterionic surfactants and/or one or more other pharmaceutically acceptable excipient(s). It might be emphasized that when the liquid vehicle (of component-2) is in the form of aqueous vehicle, then the viscosity enhancing agent is preferably present in component-2 and when the liquid vehicle (of component-2) is in the form of oily vehicle, then the viscosity enhancing agent is preferably present in component-1.

In an embodiment, the component-2 of the present invention additionally comprises of one or more substances selected from but not limited to a group comprising co-surfactants, solvents/co-solvents, water, oily component, hydrophilic solvents, preservatives, antioxidants, anti-foaming agents, stabilizers, buffering agents, pH adjusting agents, osmotic agents, isotonicity producing agents, or any other excipient soluble in the water miscible solvent known to the art or mixtures thereof. In an embodiment of the present invention, the co-surfactant is selected from but not limited to a group comprising polyethylene glycols; polyoxyethylene-polyoxypropylene block copolymers known as “poloxamer”; polyglycerin fatty acid esters such as decaglyceryl monolaurate and decaglyceryl monomyristate; sorbitan fatty acid ester such as sorbitan monostearate; polyoxyethylene sorbitan fatty acid ester such as polyoxyethylene sorbitan monooleate (TWEEN®); polyethylene glycol fatty acid ester such as polyoxyethylene monostearate; polyoxyethylene alkyl ether such as polyoxyethylene lauryl ether; polyoxyethylene castor oil and hardened castor oil, such as polyoxyethylene hardened castor oil; and the like or mixtures thereof. In an embodiment of the present invention, the solvent/cosolvent is selected from but not limited to a group comprising alcohols such as propylene glycol, polypropylene glycol, polyethylene glycol (such as PEG300, 400, 600, etc.), glycerol, ethanol, triacetin, dimethyl isosorbide, glycofurol, propylene carbonate, water, dimethyl acetamide, and the like or mixtures thereof. More preferably the solvent used is ethanol. The choice of the solvent/cosolvent and its quantity primarily depends on the solubility of the active agent(s). It might be emphasized that when the composition is formulated with a water-soluble solvent such as ethanol, the solvent will diffuse rapidly out of the injected volume leaving a high viscosity depot that is well suited for long term drug delivery. Suitable anti-foaming agents include for example silicon emulsions or sorbitan sesquoleate. Suitable stabilizers to prevent or reduce the deterioration of the other components in compositions of the present invention include antioxidants such as glycine, alpha-tocopherol or ascorbate, BHA, BHT, and the like or mixtures thereof. Suitable tonicity modifier includes for example mannitol, sodium chloride, and glucose. Suitable buffering agent includes for example acetates, phosphates, and citrates with suitable cations. It might be however understood that certain excipients used in the present composition can serve more than one purpose.

In an embodiment, the present invention provides a pharmaceutical kit suitable for in situ formation of a biodegradable depot gel or implant from the novel compositions as described herein, in the body of a subject in need thereof, which comprises a device containing tamsulosin or letrozole microparticles and optionally one or more pharmaceutical acceptable excipient(s), and a device containing liquid vehicle and optionally one or more pharmaceutical acceptable excipient(s); wherein the devices allow for expulsion of contents of the two devices for enabling mixing together prior to administration of the contents into the body of the subject.

In an embodiment, the present invention provides novel injectable depot compositions wherein the component-1 is presented as a dry powder and component-2 is presented as a liquid vehicle. The said component-1 is reconstituted with component-2 to obtain a parenteral suspension, which when injected intramuscularly or subcutaneously, forms a hydrogel or emulsigel at injection site that acts as a depot from which tamsulosin or letrozole is released in a sustained manner for prolonged time period. This helps in simplifying the available daily dosage regimen for tamsulosin or letrozole. Further, the primary barrier for the release of tamsulosin or letrozole would be the in situ hydrogel formed and the secondary barrier for release of tamsulosin or letrozole would be anticipated from the biodegradable polymeric drug microparticles or nanoparticles that leads to an effective depot thr tamsulosin or letrozole at the injection site and releases tamsulosin or letrozole in a sustained manner over an extended period of time to achieve the desired therapeutic concentration. It is an advantage of the present invention that rate of release of tamsulosin or letrozole can be dually modulated by in situ gelling composition and the biodegradable particulate form of the of tamsulosin or letrozole dispersed in the gelling composition. The term “in situ gelling composition” as used herein refers to a composition comprising a drug preferably in the form of microparticles or nanoparticles, a biodegradable polymer and optionally a viscosity enhancing agent, which is optionally reconstituted with a liquid vehicle and delivered to a patient as an injectable liquid but solidifies into a solid depot composition upon in vivo administration.

In another embodiment, the component-2 of the present invention comprises of one or more water miscible solvents or cosolvents which can get easily assimilated away from the injection site by the bodily process leaving behind the polymeric gel material at the injection site. In another aspect of the present invention, the composition of component-2 shall preferably keep the viscosity building polymeric material in unhydrated particulate form; thus preventing a viscosity build up in reconstituted suspension for injection, which in turn facilitates syringibility even at higher concentration of high viscosity building polymers used in the formulation.

In an embodiment, component-1 of the two component system relates to biodegradable microparticles or nanoparticles formulated as matrix system comprising of tamsulosin or letrozole as active agent, at least one biodegradable polymer(s), at least one hydrophilic cellulosic biocompatible polymer(s) wherein the biodegradable microparticles or nanoparticles are partially or entirely embedded and which acts as release modifier and optionally one or more pharmaceutical excipient(s), wherein the hydrophilic cellulosic biocompatible polymer upon contact with bodily fluids gets hydrated faster and forms a gel around the biodegradable microparticles or nanoparticles and later on further hydration leads the gel layer to erode followed by dissolution of hydrated entrapped cellulosic biocompatible polymer leading to formation of channels in the biodegradable microparticles or nanoparticles matrix through which drug is released. The present invention also describes a novel method of preparation of biodegradable microparticles or nanoparticles without using polyvinyl alcohol (PVA) (it may be noted that PVA is approved and listed in IIG for microsphere) as emulsion stabilizer. Emulsion stabilizer such as NaCMC, semisynthetic cellulosic polymers, gelatin and like are also useful. As the cellulosic polymer is biocompatible, it leads to an advantage of reducing the time of production of microparticles or nanoparticles by removing manufacturing steps like washing and filtration/centrifugation. Component-1 forms a readily dispersible composition upon reconstitution with suitable liquid vehicle i.e., component-2. In an embodiment the component-2 is in the form of preferably liquid vehicle for reconstitution of component-1 comprising at least one water immiscible solvent (e.g., oil) and optionally with one or more pharmaceutical acceptable excipients. In another preferred embodiment the component-2 is in the form of preferably liquid vehicle for reconstitution of component-1 comprising at least one oil, at least one surfactant and optionally with one or more pharmaceutical acceptable excipient(s). In one of the embodiment the component-2 is in the form of a liquid vehicle for reconstitution of component-1 comprising at least one water miscible solvent, optionally with one or more pharmaceutical acceptable excipient(s).

The present invention also describes a novel method of preparation of biodegradable microparticles or nanoparticles in the form of matrix by using a cellulosic biocompatible polymer having multiple properties like emulsion stabilizer, drug release modifier and a gel forming agent. In an embodiment, a cellulosic polymer such as sodium carboxymethyl cellulose (NaCMC) is used as an emulsion stabilizer during preparation of the microparticles or nanoparticles and entraps the individual microparticles or nanoparticles formed. The said polymer is approved for parenteral use and hence does not need removal. The said polymer also acts as a viscosity enhancing agent.

In an embodiment of the present invention, temperature sensitive biocompatible polymers may be used as the gel matrix, for example, a block copolymer having thermal gelation properties wherein the polymer is a gel at physiological temperatures (about 37° C.) and is a liquid above or below physiological temperatures would be functional. In the case of a gel having reverse thermal-gelation properties, the block copolymer would be a liquid at temperatures below the gelation temperature and would form a gel at above the gelation temperature. Conversely, a block copolymer having conventional thermal-gelation properties would be a liquid above the gelation temperature and a gel at or below the gelation temperature. When a biocompatible block copolymer having reverse thermal-gelation properties is employed, microparticles containing tamsulosin or letrozole can be loaded in the block copolymer which is in solution form at below physiological temperatures (about 37° C.) such as room temperature. Because such block copolymers are soluble in water when cooled, the microparticles or nanoparticles may be easily loaded within the solution. Furthermore, when administered, the block copolymer solution, once in the gel state, is able to retain the microparticles or nanoparticles.

In another embodiment, the viscosity enhancing agent(s) is present in the composition of the present invention wherein the biodegradable microparticles or nanoparticles are partially or entirely embedded and wherein the viscosity enhancing agent(s) acts as release modifier, which composition upon contact with bodily fluids gets hydrated and forms a gel around the biodegradable microparticles or nanoparticles. In an embodiment, the viscosity enhancing agent(s) is a biocompatible cellulosic polymer which acts as microparticle or nanoparticle stabilizer, active agent release modifier and/or a gel forming agent.

In another embodiment of the present invention, the composition comprising component-1 and component-2 as described herein may additionally comprise at least one another component referred to as component-3. The said third component or any further component(s) might comprise diluting fluids of carriers/vehicles or solvents which might be necessary to dilute or stabilize the injectable composition or to facilitate the desired objective of achieving a sustained release of the active agent(s) from a depot formed in situ in any manner.

In an embodiment, the present invention provides microparticles or nanoparticles of tamsulosin or letrozole consisting essentially of a matrix of a biocompatible and biodegradable polymer wherein the said microparticles or nanoparticles are reconstituted in a liquid vehicle such that they are substantially uniformly distributed; said tamsulosin or letrozole being progressively and continuously released over a period of at least 1 day when the microparticles or nanoparticles are placed in an aqueous physiological environment, with a reduced or substantially absent first phase of accelerated release.

In an embodiment of the present invention, the injectable composition additionally comprises a thermogelling polymer which is useful to formulate the microparticles or nanoparticles, wherein the said thermogelling polymer may be present within or outside or partly within and partly outside the microparticles or nanoparticles. In another embodiment of the present invention, the composition forms an in-situ gel or gel-like structure or implant which is comprised of a network of cross-linked polymeric monomers wherein the network forms intra-network aggregates in aqueous environment of the bodily fluids. In yet another embodiment, the in-situ gel responds reversibly to a change in one or more in vivo conditions such as temperature, pH, and ionic conditions. Particularly, the in situ gel is able to imbibe or solubilize a large amount of therapeutic agent and deliver a substantially linear and sustained release of therapeutic agent under physiological conditions.

In an embodiment, the present invention provides a depot composition for parenteral administration comprising tamsulosin or letrozole as active agent, a biocompatible lactic-acid based polymer; a polymer solvent that forms flowable gel with said biocompatible lactic-acid based polymer, wherein said polymer solvent is selected from the group consisting of triacetin, n-methyl-2-pyrrolidone, 2-pyrrolidone, glycerol formal, methyl acetate, benzyl benzoate, ethyl acetate, methyl ethyl ketone, dimethylformamide, dimethyl sulfoxide, tetrahydrofuran, caprolactam, decymethylsulfoxide, oleic acid, and 1-dodecylazacyclo-heptan-2-one and mixtures thereof; and an amount of an emulsifying agent dispersed in the form of a dispersed droplet phase in the flowable gel, wherein the emulsifying agent in combination with the polymer solvent renders said polymer solution thixotropic, said emulsifying agent selected from the group consisting of ethanol, isopropyl alcohol, and mixture thereof; and tamsulosin or letrozole homogenously dissolved or dispersed in the flowable gel; wherein the depot composition is adapted to release tamsulosin or letrozole for a substantially longer duration.

In an embodiment, it is an advantage of the injectable compositions of the present invention that the compositions upon reconstitution for injection are not very viscous. Often the viscosity enhancing polymer remains in substantially unhydrated form during injection facilitating easy injection using a standard gauge needle. Upon injection, the said polymer gets hydrated by bodily aqueous fluids forming a substantially thick gel at injection site and thereby creates a primary barrier for initial burst release of tamsulosin or letrozole from the biodegradable microparticles or nanoparticles and later provides a sustained release of the tamsulosin or letrozole from the biodegradable microparticle or nanoparticle system, thus providing an option for modulating the drug release so as to obtain a sustained release of the active agent(s) for an extended period of time. The inventors of the present invention with intellectual expertise have carried out undue experimentation to prepare novel injectable depot compositions which are substantially devoid of so called ‘burst’ release of the active agent thus providing a sustained release of the active agent(s) for an extended period of time.

The compositions of the present invention are sufficiently stable so that a depot comprising one quantity or batch of the composition can provide continuous release of the composition to a patient or subject for up to about six months. The release of the active agent is over alternative periods of time, such as up to about one week, up to about two weeks, up to about three weeks, up to about one month, up to about two months, up to about three months, up to about four months, or up to about five months, or up to about six months.

The use of a combination of two or more different implant or microparticle formulations according to the present invention enables a wide range of release profiles to be achieved by appropriate selection of polymers and/or loading of the active agent tamsulosin or letrozole into the microparticles. This may be advantageous for the treatment of certain diseases. For example, it may be desirable to provide a high initial dose of tamsulosin or letrozole, followed by a lower dose for the remainder of the treatment. This may be achieved by selecting a first implant or microparticle formulation which has a high initial release rate of tamsulosin or letrozole and a second implant or microparticle formulation which has a more constant release rate. The cumulative tamsulosin or letrozole release from the two formulations thereby provides a high initial dose followed by a substantially constant release rate for the remainder of the treatment period. Alternatively, by appropriate selection of two or more different implant or microparticle formulations it is possible to provide a cumulative release of tamsulosin or letrozole which is substantially zero order (i.e. substantially constant) throughout the treatment period. The release profile of tamsulosin or letrozole from the first and second implants/microparticle formulations may be controlled by, for example, varying the lactide:glycolide ratio and/or the molecular weight of the polylactide or poly(lactide-co-glycolide) and/or the loading of tamsulosin or letrozole in the implant and/or the amount of the viscosity enhancing polymer.

In yet another embodiment of the present invention is provided process for preparation of such novel injectable compositions which comprises preparation of tamsulosin or letrozole microparticles or nanoparticles and optionally a liquid vehicle in which the said microparticles or nanoparticles may be reconstituted prior to administration.

In a further embodiment, the process for preparation of compositions according to the present invention comprises of the following steps:
i) dissolving tamsulosin or letrozole and biodegradable polymer(s) in an water immiscible solvent and emulsification with water containing an emulsifier,
ii) removing the solvent leading to formation of microparticles or nanoparticles,
iii) mixing the microparticles or nanoparticles of step (ii) optionally with viscosity enhancing agent(s) and/or optionally with one or more excipient(s) to form component-1,
iv) mixing the liquid vehicle optionally with viscosity enhancing agent(s) and/or other excipients to form component-2, and
v) mixing the component-1 and component-2 to obtain the desired composition before administration.

In a further embodiment, the process for preparation of compositions according to the present invention comprises of the following steps:
i) dissolving or dispersing the active agent tamsulosin or letrozole and biodegradable polymer(s) in a water immiscible solvent,
ii) homogenizing the solution of step (i) with an aqueous emulsifier solution, evaporating the solvent to form the microparticles or nanoparticles, washing and freeze drying the aqueous dispersion of microparticles or nanoparticles,
iii) mixing the microparticles or nanoparticles of step (ii) optionally with viscosity enhancing agent(s) and/or optionally with one or more excipient(s) to form component-1,
iv) mixing the liquid vehicle optionally with viscosity enhancing agent(s) and/or other excipient(s) to form component-2, and
v) mixing the component-1 and component-2 to obtain the desired composition before administration.

In a further embodiment, the process for preparation of compositions according to the present invention comprises of the following steps:
i) dissolving or dispersing the active agent and biodegradable polymer(s) in an appropriate solvent and spray drying to form microparticles or nanoparticles,
ii) mixing the microparticles or nanoparticles of step (i) optionally with viscosity enhancing agent(s) to form component-1,
iii) mixing the liquid vehicle optionally with viscosity enhancing agent(s) and/or other excipient(s) to form component-2, and
iv) mixing the component-1 and component-2 to obtain a suitable injectable dosage form composition before administration.

In a further embodiment, the inventors of the present invention had found that during the process of preparation of the microparticles or nanoparticles, when homogenization was done preferably using Ultra Turrax homogeniser for a particular time period such as for about 30 seconds at a specific speed such as about 15000 rpm, the microparticles obtained had better shape and properties. Further, the washing of microparticles or nanoparticles when carried out by repeated centrifugation and resuspension of the residue in fresh water for injection to remove the solvent and emulsifier, produced very good microparticles or nanoparticles that were appreciably hard, had good shape and were substantially non-porous. It might be understood that the use of a suitable homogenizer and optimized process parameters such as pressure, number of cycles, flow rate of the feed, and the like for the preparation of emulsion shall produce microparticles having defined particle size, shape and other desirable characteristics. Homogenization was also done by vigorous stirring of both the phases by using a magnetic stirrer or over head stirrers with anchor or paddle stirring element. During emulsification stage, the variables like speed of stirring, shape and dimension of stirring element and vessel with reference to batch size would be precisely controlled to yield microparticles of desired shape and size. It is also desired that washing of the formed microparticles shall be carried out using cross flow or tangential flow filtration system (Minimate® TFF system from Pall Corporation), wherein the microparticles suspension is concentrated by filtration and diluted with fresh water repeatedly to wash the microparticles.

In an embodiment of the present invention, the process parameters employed during the preparation of biodegradable microparticles or nanoparticles is intended to achieve the production of the microparticles or nanoparticles with defined shape, size distribution and quantity of active pharmaceutical agent entrapped in polymer matrix in a substantially reproducible manner. In a preferred embodiment, the process employed in the present invention to produce the microparticles or nanoparticles as by w/o, o/w, w/o/w and o/w/o more preferably o/w emulsion is solvent evaporation technique known to the art. The different ingredients used to produce microparticles or nanoparticles in the present invention are selected from the commonly used compounds known to the art.

In a further embodiment, in the o/w emulsion technique, the active agent(s) and the biodegradable polymer(s) were dissolved in water immiscible solvents considered as ‘oil phase’; the solution was homogenized with a ‘water phase’ containing pharmaceutically acceptable emulsifier. The resultant emulsion was stirred optionally with moderate heating optionally under applied vacuum so that the inner organic solvent was evaporated during agitation leaving behind the suspension of microparticles or nanoparticles formed due to hardening of biodegradable polymers from oil phase. Both the emulsifier and the organic solvents used were lost during the process and hence not present in final product or present within acceptable limits. In a process of present invention, the organic solvent was removed by evaporation through agitation or warming, and emulsifier was removed by washing with water. Further, the emulsifier enhances stabilization of oil droplets against coalescence. Emulsifier concentration in the water phase strongly influences drug distribution within microparticles and release profiles. Further, emulsifier was added optionally to the water phase in order to keep the precipitating biodegradable polymer as fine independent dispersed particles.

In another embodiment of the present invention, the biodegradable microparticles or nanoparticles are produced by spray drying or lyophilization technique. In order to obtain the desired microparticles or nanoparticles, appropriate quantity of cryoprotectants is used in the composition to facilitate ready dispersibility of the composition in the diluent (vehicle) for reconstitution. Cryoprotectants such as lactose, trehalose, sucrose, or mannitol are preferably incorporated into the composition along with the biodegradable drug microparticulate form at the time of spray drying or lyophilization.

In an embodiment of the present invention, the microparticles are preferably spherical shaped. The mean particle size of microparticles is in the range of about 1 to about 250 microns, preferably about 2 to about 150 microns, and more preferably about 10 to about 100 microns as measured by a suitable technique known to the art, whereby administration of the microparticles to a subject can be carried out with a standard gauge needle. It was also observed that narrower the particle size distribution range, better was the redispersibility of microparticles in the liquid vehicle, and better was reproducibility of drug release pattern from the microparticles. In an embodiment, the injectable composition of the present invention is in the form of nanoparticles comprising active agent(s) preferably having a mean particle size range of about 100 nm to about 2000 nm, wherein said nanoparticles are suspended in a vehicle and targeted for delivery to specific site of disease to provide a sustained release of active agent(s) for an extended time period.

In an embodiment, the composition of the present invention is preferably in the form of parenteral composition which can be administered to a subject, animals or humans, preferably via intramuscular, intradermal, cutaneous or subcutaneous routes. Specifically the parenteral composition according to the invention can be given by any of the following routes such as among others: intra-abdominal, intra-articular, intra-capsular, intra-cervical, intra-cranial, intra-ductal, intra-dural, intra-lesional, intra-ocular, intra-locular, intra-mural, intra-operative, intra-parietal, intra-peritoneal, intra-plural, intra-pulmonary, intra-spinal, intrathoracic, intra-tracheal, intra-tympanic, intra-uterine or transdermal. In a preferred embodiment, the composition of the present invention is in the form of parenteral composition, which may be administered via intramuscular or subcutaneous route.

In an embodiment, the in-situ gelling composition according to the present invention can deliver tamsulosin or letrozole directly to the target and provide short or long-term treatment by the controlled release of tamsulosin or letrozole in the target area. The application of the composition may be by any means necessary to introduce tamsulosin or letrozole in vivo into a subject such as a mammal including invasive surgery and/or application, preferentially, by injection. The parenteral route for delivering the compositions of the present invention is selected from the group consisting of subcutaneous, intramuscular, intraorbital, intracapsular, intraspinal, intrasternal, or the like. The depot formed in vivo is of a consistency selected from the group consisting of a viscous material, a gel or semi-solid, and combinations thereof. The rate of release of tamsulosin or letrozole from the depot might vary based on variation in one or more factors such as initial particle size, levels of gel in the formulation, the amount of active agent, levels of any additional materials in the formulation, the subject, subject metabolism, the administration site, and combinations thereof.

In an essential embodiment, the depot formed by the composition of the present invention traps the tamsulosin or letrozole microparticles or nanoparticles within the depot in a relatively short period of time such that any free microparticle or nanoparticle is substantially captured by the coagulating process before being carried away from the depot. For the purposes of this specification ‘depot’ is defined as a substance (preferably containing an active agent) that is retained in close proximity to the site of injection so that release of the active agent occurs over a prolonged period of time. In an embodiment, the depot erodes/dissolves in the in vivo environment of the subject over time and in doing so releases the active agent into the subject. A further advantage of the present invention is that leakage from the injection site is minimized or removed altogether. The gelling characteristics of the formulation bind the tamsulosin or letrozole microparticles or nanoparticles within close proximity of the injection site. This avoids back flow of formulation out through the injection point thus stopping unwanted waste of the agent and also gives a clean wound/administration area. In addition, the combination of microparticle or nanoparticle and polymeric delivery systems also increases design flexibility of the drug delivery system to allow a fit to individual needs. Such drug delivery systems have modified or improved release profiles and individual delivery system through modulating the drug dissolution rate and gel matrix erosion rate.

In yet another embodiment of the present invention is provided a method of forming a depot gel or an implant in situ, in a living body, which comprises preparing an in situ gelling formulation according to the method described herein, placing the formulation within the body and allowing the liquid vehicle to disperse or dissipate to produce a solid or gel implant.

In yet another embodiment of the present invention is provided use of an in situ gelling formulation as described herein in the manufacture of a medicament for the treatment of a condition treatable by tamsulosin or letrozole in a mammal particularly a human being.

In yet another embodiment of the present invention is provided a method of using the compositions of tamsulosin or letrozole according to the present invention which comprises administering to a subject/patient in need thereof an effective amount of the said composition. The novel compositions of the present invention comprising tamsulosin is particularly useful for management such as prophylaxis, amelioration and/or treatment of subjects for the signs and symptoms of benign prostatic hyperplasia, and compositions of the present invention comprising letrozole is particularly useful for management such as prophylaxis, amelioration and/or treatment of hormonally-responsive breast cancer. In still another embodiment is provided use of the composition according to the present invention comprising tamsulosin as the active agent for the manufacture of a medicament for the prophylaxis, amelioration and/or treatment of moderate to severe symptoms of benign prostatic hyperplasia. In another embodiment is provided the use of a composition according to the present invention comprising letrozole as the active agent for the manufacture of a medicament for the prophylaxis, amelioration and/or treatment of hormonally-responsive breast cancer.

The above-described exemplary embodiments are intended to be illustrative in all respects, rather than restrictive, of the present invention. Thus the present invention is capable of many variations in detailed implementation that can be derived from the description contained herein by a person skilled in the art. All such variations and modifications are considered to be within the scope and spirit of the present invention.

### EXAMPLES

### Example-1

| | | |
|---|---|---|
| S. No. | Ingredient | Quantity/unit dose |

Component-1

| | | | |
|---|---|---|---|
| 1. | Tamsulosin | 12.0 | mg |
| 2. | Poly (lactide-co-glycolide) 50/50 | 400.0 | mg |
| 3. | Polyvinyl alcohol | 240.0 | mg (lost in processing) |
| 4. | Dichloromethane | 10.0 | ml (lost in processing) |
| 5. | Water for injection | 24.0 | ml (lost in processing) |
| 6. | Hydroxyethylcellulose | 40.0 | mg |

Component-2

| | | | |
|---|---|---|---|
| 7. | Polyethylene glycol | 1.5 | ml |
| 8. | Glycerin | 0.5 | ml |

### Procedure:

i) Polyvinyl alcohol solution was prepared by dissolving Polyvinyl alcohol in hot Water for injection under stirring.
ii) Tamsulosin and biodegradable polymer are dissolved in Dichloromethane and added to Polyvinyl alcohol solution under homogenization.
iii) The emulsion of step (ii) was stirred and optionally vacuum applied until Dichloromethane was completely removed leaving behind the suspension of microparticles.
iv) Microparticles were washed with water for injection to remove Polyvinyl alcohol
v) Residue of step (iv) was resuspended in water for injection and lyophilized to obtain powder of microparticles of Tamsulosin entrapped in Poly(lactide-co-glycolide) 50/50
vi) The prepared microparticles of step (v) were blended with Hydroxy ethylcellulose and filled in suitable vial or prefilled syringe (component-1).
vii) Component-2 was prepared by mixing Polyethylene glycol and Glycerin and filled in a vial.

### Example-2

| | | |
|---|---|---|
| S. No. | Ingredient | Quantity/unit dose |

Component-1

| | | | |
|---|---|---|---|
| 1. | Tamsulosin | 12.0 | mg |
| 2. | Poly (lactide-co-glycolide) 50/50 | 120.0 | mg |
| 3. | Gelatin | 30.0 | mg (lost in processing) |
| 4. | Dichloromethane | 2.0 | ml (lost in processing) |
| 5. | Water for injection | 5.0 | ml (lost in processing) |
| 6. | Mannitol | 7.0 | mg |
| 7. | Sodium carboxymethyl cellulose | 60.0 | mg |

Component-2

| | | | |
|---|---|---|---|
| 8. | Propylene glycol | 1.7 | ml |
| 9. | Ethanol | 0.3 | ml |

### Procedure:

i) A solution was prepared by dissolving gelatin in warm (40° C.) Water for injection under stirring and cooling to room temperature by continuous stirring.
ii) Tamsulosin and Poly (lactide-co-glycolide) 50/50 were dissolved in Dichloromethane and the clear solution was added to gelatin solution under homogenization.
iii) The emulsion of step (ii) was stirred until Dichloromethane was completely evaporated leaving behind the suspension of microparticles.
iv) The microparticles of step (iii) were washed with water to remove gelatin. The washing was carried out by repeated centrifugation at about 5° C. and resuspending the residue in fresh water for injection.
v) The finally obtained residue was dispersed in Mannitol solution, lyophilized to get free flowing powder of microparticles of Tamsulosin entrapped in Poly (lactide-co-glycolide).
vi) The prepared microparticles were blended with Sodium carboxymethyl cellulose and filled in suitable vial or prefilled syringe (component-1).
vii) Component-2 was prepared by mixing Propylene glycol & ethanol, and filled in vial.

### Example-3

| | | |
|---|---|---|
| S. No. | Ingredient | Quantity/unit dose |

Component-1

| | | | |
|---|---|---|---|
| 1. | Tamsulosin | 12.0 | mg |
| 2. | Poly (lactide-co-glycolide) 50/50 | 120.0 | mg |
| 3. | Polyvinyl alcohol | 30.0 | mg (lost in processing) |
| 4. | Dichloromethane | 2.0 | ml (lost in processing) |
| 5. | Water for injection | 5.0 | ml (lost in processing) |
| 6. | Mannitol | 7.0 | mg |

Component-2

| | | | |
|---|---|---|---|
| 7. | Sodium carboxymethyl cellulose | 50.0 | mg |
| 8. | Propylene glycol | 2.2 | ml |
| 9. | Ethanol | 0.3 | ml |

### Procedure:

i) A solution was prepared by dissolving Polyvinyl alcohol in hot Water for injection under stirring and cooling to room temperature by continuous stirring.
ii) Tamsulosin and Poly (lactide-co-glycolide) 50/50 were dissolved in Dichloromethane and the clear solution was added to gelatin solution under homogenization.
iii) The emulsion of step (ii) was stirred until Dichloromethane was completely evaporated leaving behind the suspension of microparticles.
iv) The microparticles of step (iii) were washed with water to remove gelatin.
v) The finally obtained residue was dispersed in Mannitol solution, lyophilized to get free flowing powder of microparticles of Tamsulosin entrapped in Poly (lactide-co-glycolide).
vi) The prepared microparticles were filled in vial or prefilled syringe (component-1).
vii) Component-2 was prepared by mixing Propylene glycol & ethanol, followed by mixing with Sodium carboxymethyl cellulose by stirring, and filled in vial.

### Example-4

| | | |
|---|---|---|
| S. No. | Ingredient | Quantity/unit dose |

Component-1

| | | | |
|---|---|---|---|
| 1. | Tamsulosin | 12.0 | mg |
| 2. | Poly (glycolic acid) | 400 | mg |
| 3. | Polyvinyl alcohol | 60.0 | mg (lost in processing) |
| 4. | Dichloromethane | 3.0 | ml (lost in processing) |
| 5. | Water for injection | 6.0 | ml (lost in processing) |
| 6. | Hydroxypropyl methylcellulose | 45.0 | mg |

Component-2

| | | | |
|---|---|---|---|
| 7. | Propylene glycol | 1.2 | ml |
| 8. | Glycerin | 0.6 | ml |
| 9. | Phosphate buffer saline pH7.4 | 0.2 | ml |

### Procedure:

i) Polyvinyl alcohol solution was prepared by dissolving Polyvinyl alcohol in Water for injection at 90° C. under stirring and cooling to room temperature.
ii) Tamsulosin and Poly (glycolic acid) were dissolved in Dichloromethane and the clear solution was added to Polyvinyl alcohol solution under homogenization.
iii) The emulsion of step (ii) was stirred until Dichloromethane was completely evaporated leaving behind the suspension of microparticles.
iv) Microparticles of step (iii) were washed with water for injection to remove Polyvinyl alcohol.
v) The finally obtained residue was lyophilized to get free flowing powder of microparticles of Tamsulosin entrapped in Poly (glycolic acid).
vi) The microparticles of step (v) were blended with Hydroxypropyl methylcellulose and filled in suitable vial or prefilled syringe (component-1).
vii) Component-2 was prepared by mixing Propylene glycol, Glycerin and Saline pH 7.4 Phosphate buffered, and filled in a vial.

### Example-5

| | | |
|---|---|---|
| S. No. | Ingredient | Quantity/unit dose |

Component-1

| | | | |
|---|---|---|---|
| 1. | Tamsulosin | 12.0 | mg |
| 2. | D,L Poly (lactide) | 400 | mg |
| 3. | Polyvinyl alcohol | 60.0 | mg (lost in processing) |
| 4. | Trichloroethane | 3.0 | ml (lost in processing) |
| 5. | Water for injection | 6.0 | ml (lost in processing) |
| 6. | Hydroxyethyl cellulose | 23.0 | mg |

Component-2

| | | | |
|---|---|---|---|
| 7. | Methyl cellulose | 22.0 | mg |
| 8. | Propylene glycol | 1.2 | ml |
| 9. | Glycerin | 0.6 | ml |

### Procedure:

i) Polyvinyl alcohol solution was prepared by dissolving Polyvinyl alcohol in Water for injection at 90° C. under stirring and cooling to room temperature.
ii) Tamsulosin and D,L Poly (lactide) were dissolved in Trichloroethane and the clear solution was added to Polyvinyl alcohol solution under homogenization.
iii) The emulsion of step (ii) was stirred until Trichloroethane was completely evaporated leaving behind the suspension of microparticles.
iv) Microparticles were washed with water for injection to remove Polyvinyl alcohol.
v) The finally obtained residue was lyophilized to get free flowing powder of microparticles of Tamsulosin entrapped in Poly (lactide).
vi) The microparticles of step (v) were blended with Hydroxyethyl cellulose and filled in suitable vial or prefilled syringe (component-1).
vii) Component-2 was prepared by mixing Propylene glycol, Glycerin and Methyl cellulose, and filled in a vial.

### Example-6

| | | |
|---|---|---|
| S. No. | Ingredient | Quantity/unit dose |

Component-1

| | | | |
|---|---|---|---|
| 1. | Letrozole | 20.0 | mg |
| 2. | Poly(lactide-co-glycolide) | 200.0 | mg |
| 3. | Polyvinyl alcohol | 30.0 | mg (lost in processing) |
| 4. | Dichloromethane | 40.0 | ml (lost in processing) |
| 5. | Water for injection | 80.0 | ml (lost in processing) |
| 6. | Hydroxypropyl methyl cellulose | 30.0 | mg |

Component-2

| | | | |
|---|---|---|---|
| 7. | Propylene glycol | 1.4 | ml |
| 8. | Glycerin | 0.4 | ml |
| 9. | Ethanol | 0.2 | ml |

### Procedure:

i) Polyvinyl alcohol solution was prepared by dissolving Polyvinyl alcohol in hot Water for injection under continuous stirring followed by cooling.
ii) Letrozole and Poly(lactide-co-glycolide) were dissolved in Dichloromethane and the clear solution was added to Polyvinyl alcohol solution under homogenization.
iii) The emulsion of step (ii) was stirred until Dichloromethane was completely evaporated leaving behind the suspension of microparticles.
iv) The microparticles were washed with water for injection to remove Polyvinyl alcohol.
v) The finally obtained residue of step (iv) was lyophilized to get free flowing powder of microparticles of Letrozole entrapped in Poly(lactide-co-glycolide).
vi) The microparticles of step (v) were blended with Hydroxypropyl methyl cellulose and filled in suitable vial or prefilled syringe (component-1).
vii) Component-2 was prepared by mixing Propylene glycol, Glycerin and Ethanol.

### Example-7

| | | |
|---|---|---|
| S. No. | Ingredient | Quantity/unit dose |

Component-1

| | | | |
|---|---|---|---|
| 1. | Letrozole | 20.0 | mg |
| 2. | Poly (Sebacic acid) | 110.0 | mg |
| 3. | Gelatin | 30.0 | mg (lost in processing) |
| 4. | Chloroform | 2.0 | ml (lost in processing) |
| 5. | Water for injection | 5.0 | ml (lost in processing) |
| 6. | Mannitol | 7.0 | mg |
| 7. | Sodium carboxymethyl cellulose | 40.0 | mg |

Component-2

| | | | |
|---|---|---|---|
| 8. | Polyethylene glycol | 1.5 | ml |
| 9. | Ethanol | 0.5 | ml |

### Procedure:

i) A solution was prepared by dissolving gelatin in hot Water for injection under stirring and cooling to room temperature by continuous stirring.
ii) Letrozole and Poly (Sebacic acid) were dissolved in Chloroform and the clear solution was added to gelatin solution under homogenization using Ultra Turrax homogeniser
iii) The emulsion of step (ii) was stirred with a suitable stirrer at ambient temperature until Chloroform was completely evaporated leaving behind the suspension of microparticles.
iv) The microparticles of step (iii) were washed with water to remove gelatin.
v) The finally obtained residue was dispersed in Mannitol solution, lyophilized to get free flowing powder of microparticles of Letrozole entrapped in Poly (Sebacic acid).
vi) The prepared microparticles were blended with Sodium carboxymethyl cellulose and filled in suitable vial or prefilled syringe (component-1).
vii) Component-2 was prepared by mixing Polyethylene glycol and ethanol, and filled in a vial.

### Example-8

| | | |
|---|---|---|
| S. No. | Ingredient | Quantity/unit dose |

Component-1

| | | | |
|---|---|---|---|
| 1. | Letrozole | 20.0 | mg |
| 2. | Poly (lactide-co-glycolide) 50/50 | 120.0 | mg |
| 3. | Gelatin | 30.0 | mg (lost in processing) |
| 4. | Dichloromethane | 2.0 | ml (lost in processing) |
| 5. | Water for injection | 5.0 | ml (lost in processing) |
| 6. | Mannitol | 7.0 | mg |
| 7. | Sodium carboxymethyl cellulose | 50.0 | mg |

Component-2

| | | | |
|---|---|---|---|
| 8. | Peanut oil | 1.8 | ml |
| 9. | Polyoxyl 35 castor oil | 0.2 | ml |

### Procedure:

i) A solution was prepared by dissolving gelatin in Water for injection under stirring and cooling to room temperature by continuous stirring.
ii) Letrozole and Poly (lactide-co-glycolide) 50/50 were dissolved in Dichloromethane and the clear solution was added to gelatin solution under homogenization.
iii) The emulsion of step (ii) was stirred until Dichloromethane was completely evaporated leaving behind the suspension of microparticles.
iv) The microparticles of step (iii) were washed with water to remove gelatin.
v) The finally obtained residue was dispersed in Mannitol solution, lyophilized to get free flowing powder of microparticles of Letrozole entrapped in Poly (lactide-co-glycolide).
vi) The microparticles were filled in vial or prefilled syringe (component-1).
vii) Component-2 was prepared by mixing Peanut oil and Polyoxyl 35 castor oil.

### Example-9

| | | |
|---|---|---|
| S. No. | Ingredient | Quantity/unit dose |

Component-1

| | | | |
|---|---|---|---|
| 1. | Letrozole | 20.0 | mg |
| 2. | Poly D, L (Lactide acid) | 110.0 | mg |
| 3. | Sodium carboxymethyl cellulose | 30.0 | mg |
| 4. | Dichloromethane | 2.0 | ml (lost in processing) |
| 5. | Water for injection | 5.0 | ml (lost in processing) |
| 6. | Mannitol | 7.0 | mg |

Component-2

| | | | |
|---|---|---|---|
| 7. | Polyethylene glycol | 1.5 | ml |
| 8. | Ethanol | 0.5 | ml |

### Procedure:

i) A solution was prepared by dissolving Sodium carboxymethyl cellulose in Water for injection under stirring and cooling to room temperature by continuous stirring.
ii) Letrozole and Poly D, L (Lactide acid) were dissolved in Dichloromethane and the clear solution was added to Sodium carboxymethyl cellulose solution under homogenization.
iii) The dispersion of droplets of step (ii) was stirred until Dichloromethane was completely evaporated leaving behind the suspension of microparticles.
iv) The finally obtained microparticle was dispersed in Mannitol solution, lyophilized to get free flowing powder of microparticles of Letrozole entrapped in Poly D, L (Lactide acid) & covered with a layer of Sodium carboxymethyl cellulose.
v) Prepared microparticles were filled in suitable vial or prefilled syringe (component-1).
vi) Component-2 was prepared by mixing Polyethylene glycol and ethanol and filled in a vial.

### Example-10

| | | |
|---|---|---|
| S. No. | Ingredient | Quantity/unit dose |

Component-1

| | | | |
|---|---|---|---|
| 1. | Tamsulosin | 12.0 | mg |
| 2. | PLGA (75:25) | 240.0 | mg |
| 3. | Gelatin | 30.0 | mg (lost in processing) |
| 4. | Dichloromethane | 2.0 | ml (lost in processing) |
| 5. | Water for injection | 5.0 | ml (lost in processing) |
| 6. | Mannitol | 7.0 | mg |

Component-2

| | | | |
|---|---|---|---|
| 7. | Polaxomer 188 | 400.0 | mg |
| 8. | Water for injection | 2.0 | ml |

### Procedure:

i) A solution was prepared by dissolving gelatin in Water for injection under stirring and cooling to room temperature by continuous stirring.
ii) Letrozole and Poly (lactide-co-glycolide) 50/50 were dissolved in Dichloromethane and the clear solution was added to gelatin solution under homogenization.
iii) The emulsion of step (ii) was stirred until Dichloromethane was completely evaporated leaving behind the suspension of microparticles.
iv) The microparticles of step (iii) were washed with water to remove gelatin.
v) The finally obtained residue was dispersed in Mannitol solution, lyophilized to get free flowing powder of microparticles of Letrozole entrapped in Poly (lactide-co-glycolide)
vi) Prepared microparticles were filled in suitable vial or prefilled syringe (component-1).
vii) Component-2 was prepared by mixing Polaxomer 188 and Water for injection & filled in a separate vial.

1-36. (canceled)

37. A novel injectable composition comprising an active agent which is tamsulosin or letrozole or its pharmaceutically acceptable salts, derivatives, isomers, polymorphs, solvates, hydrates, analogues, enantiomers, tautomeric forms or mixtures thereof, at least one biodegradable polymer(s) and optionally one or more pharmaceutically acceptable excipient(s), wherein the composition is formulated as biodegradable microparticles or nanoparticles which can be optionally reconstituted with an aqueous, hydro-alcoholic or oily liquid vehicle prior to administration, and wherein the said composition provides a prolonged release of tamsulosin or letrozole for extended periods of time.

38. A composition according to claim 37, which is in the form of an in situ gelling composition or a implant composition and which forms a depot upon administration in vivo upon contact with body fluids therefore providing a prolonged release of the active agent for extended periods of time.

39. A composition according to claim 37, comprising tamsulosin or letrozole as active agent and at least one biodegradable polymer(s), wherein the ratio of active agent to the biodegradable polymer(s) is between about 1:100 to about 100:1.

40. A composition according to claim 37, comprising tamsulosin or letrozole as active agent in an amount of from about 0.1% w/w to about 95% w/w, at least one biodegradable polymer(s) in an amount of from about 0.1% w/w to about 95% w/w, optionally one or more pharmaceutically acceptable excipient(s) in an amount of from about 0.1% to about 99.8% w/w based upon the total weight of the formulation, wherein the biodegradable polymer(s) is a polylactide polymer or a polyglycolide polymer or a poly(lactide-co-glycolide) co-polymer having an average molecular weight of from about 1,000 daltons to about 200,000 daltons; and wherein the said composition forms a gel or implant when placed in an aqueous physiological-type environment and releases the active agent for over a period of at least 7 days.

41. A composition according to claim 37, wherein the mean particle size of microparticles is in the range of about 1 to about 250 microns and the mean particle size of the nanoparticles is in the range of about 100 nm to about 2000 nm.

42. A composition according to claim 37, wherein the composition is in the form of a multi-component system comprising at least two components, component-1 and component-2.

43. A novel injectable depot composition of tamsulosin or letrozole according to claim 42 comprising of two components, wherein component-1 is in the form of a readily dispersible composition preferably microparticles or nanoparticles comprising tamsulosin or letrozole and at least one biodegradable polymer(s), optionally with one or more pharmaceutical acceptable excipient(s); and wherein component-2 is in the form of a liquid vehicle for reconstitution of component-1 comprising at least one water miscible or water immiscible solvent, optionally with one or more pharmaceutical acceptable excipient(s); and wherein the compositions comprise at least one viscosity enhancing agent(s) in an amount between about 0.1% to about 50% by weight of the composition either present in component-1 or component-2 or both.

44. A composition according to claim 43, wherein the viscosity enhancing agent(s) is present in an unhydrated form.

45. A composition according to claim 43, wherein the biodegradable microparticles or nanoparticles are partially or entirely embedded in the viscosity enhancing agent(s) which acts as release modifier upon contact with body fluids by getting hydrated and forming a gel around the biodegradable microparticles.

46. A composition according to claim 37, wherein the biodegradable polymer is selected from a group comprising lactic acid-based polymers; glycolic acid-based polymers; poly (D,L-lactide-co-glycolide); polycaprolactones; polyanhydrides; poly(Sebacic acid); poly(Ricenolic acid); poly(Fumaric acid); poly(Fatty acid dimmer); poly(terephthalic acid); polyamines; poly(isophthalic acid); poly(p-{carboxyphenoxy}methane); poly(p-{carboxyphenoxy}propane); poly(p-{carboxyphenoxy}hexane); polyurethanes; polyesteramides; polyorthoesters; polydioxanones; polyhydroxybutyrates; polyalkyene oxalates; polyamides; polyesteramides; polyurethanes; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic celluloses; polyacrylic acids; polybutyric acid; polyvaleric acid; polyethylene glycol; polyhydroxycellulose; chitin; chitosan; polyorthoesters and copolymers, terpolymers; dimethyl isosorbide; lipids such as cholesterol, lecithin; poly(glutamic acid-co-ethyl glutamate) and the like, or mixtures thereof.

47. A composition according to claim 46, wherein the lactic acid-based polymer is polylactide or poly (D, L-lactide-co-glycolide).

48. A composition according to claim 47, wherein the poly (D, L-lactide-co-glycolide) polymer has a monomer ratio of lactic acid to glycolic acid in the range of 100:0 to about 10:90 and an average molecular weight of from about 1,000 to 200,000 daltons.

49. A composition according to claim 43, wherein the component-1 additionally comprises excipients selected from a group comprising channel forming agents, oily components, emulsifiers, preservatives, antioxidants, stabilizers or mixtures thereof.

50. A composition according to claim 49, wherein the emulsifier is selected from a group comprising polyoxyethylene sorbitan fatty acid esters; sorbitan fatty acid esters; polysorbates, polyvinyl alcohol, polyvinyl pyrrolidone, gelatin, lecithin, polyoxyethylene castor oil derivatives; tocopherol; tocopheryl polyethylene glycol succinate; tocopherol palmitate and tocopherol acetate; cellulosic polymer, Polyoxyethylene-polyoxypropylene co-polymers, or mixtures thereof.

51. A composition according to claim 49, wherein the channel forming agent is selected from a group comprising polyglycols, ethyl vinyl alcohols, glycerin, pentaerythritol, polyvinyl alcohols, polyvinyl pyrrolidone, vinyl pyrrolidone, N-methylpyrrolidone, polysaccharides, saccharides, sugar alcohols, or mixtures thereof.

52. A composition according to claim 43, wherein the viscosity enhancing agent is selected from a group comprising cellulose derivatives, vinyl polymers, polyoxyethylene-polyoxypropylene polymers or co-polymers, polysaccharides, proteins, poly(ethyleneoxide), acrylamide polymers, polyhydroxy acids, polyanhydrides, polyorthoesters, polyamides, polycarbonates, polyalkylenes, polyalkylene glycols, polyalkylene oxides, polyalkylene terepthalates, polyvinyl alcohols, polymethacrylic acid, polyvinyl pyrrolidone and polyvinyl alcohol, polyvinyl ethers, polyvinyl esters, polyvinyl halides, polyvinylpyrrolidone, polysiloxanes, polyvinyl acetates, polystyrene, polyurethanes, synthetic celluloses, polyacrylic acids, polybutyric acid, polyvaleric acid, poly(lactide-co-caprolactone), and copolymers, derivatives; or mixtures thereof.

53. A composition according to claim 52, wherein the viscosity enhancing agent is sodium carboxymethyl cellulose or methyl cellulose.

54. A composition according to claim 43, wherein the liquid vehicle (of component-2) is in the form of an aqueous vehicle comprising water and optionally water miscible solvent selected from a group comprising a water-miscible alcohol; dimethylsulfoxide; dimethylformamide; dimethylacetamide; NMP, benzyl alcohol, a water-miscible ether; a water-miscible nitrile; a water-miscible ketone; an amide; propylene glycol; glycerin; polyethylene glycol 400; glycofurol (tetraglycol); or mixtures thereof.

55. A composition according to claim 54, wherein the water miscible solvent is selected from a group comprising glycerin, ethanol, propylene glycol and polyethylene glycols, or mixtures thereof.

56. A composition according to claim 43, wherein the liquid vehicle is an oily vehicle comprising at least one oily component selected from a group comprising vegetable oils such as corn oil, almond oil, sunflower oil, castor oil, and the like, or a lipophilic compound such as dimethyl isosorbide.

57. A composition according to claim 43, wherein the component-2 additionally comprises one or more of co-surfactants, co-solvents, hydrophilic solvents, preservatives, antioxidants, anti-foaming agents, stabilizers, buffering agents, pH adjusting agents, osmotic agents, isotonicity producing agents, or mixtures thereof.

58. A composition according to claim 37, wherein the composition additionally comprises a thermogelling or hydrogelling polymer.

59. A composition according to claim 37, which can be administered to a subject through the intramuscular, intradermal, cutaneous or subcutaneous, intra-abdominal, intra-articular, intra-capsular, intra-cervical, intra-cranial, intra-ductal, intra-dural, intra-lesional, intra-ocular, intra-locular, intra-mural, intra-operative, intra-parietal, intra-peritoneal, intra-plural, intra-pulmonary, intra-spinal, intrathoracic, intra-tracheal, intra-tympanic, intra-uterine or transdermal route.

60. A process for the preparation of injectable composition according to claim 37, which comprises preparation of tamsulosin or letrozole microparticles or nanoparticles and optionally a liquid vehicle in which the said microparticles or nanoparticles may be reconstituted prior to administration.

61. A process for the preparation of injectable composition according to claim 37, which comprises following steps:
i) dissolving tamsulosin or letrozole and biodegradable polymer(s) in an water immiscible solvent and emulsification with water containing an emulsifier,
ii) removing the solvent leading to formation of microparticles or nanoparticles,
iii) mixing the microparticles or nanoparticles of step (ii) optionally with viscosity enhancing agent(s) and/or optionally with one or more excipient(s) to form component-1,
iv) mixing the liquid vehicle optionally with viscosity enhancing agent(s) and/or other excipients to form component-2, and
v) mixing the component-1 and component-2 to obtain the desired composition before administration.

62. A process for the preparation of injectable composition according to claim 37, which comprises of the following steps:
i) dissolving or dispersing the active agent(s) and biodegradable polymer(s) in a water immiscible solvent,
ii) homogenizing the solution of step (i) with an aqueous emulsifier solution, evaporating the solvent to form the microparticles or nanoparticles, washing and freeze drying the microparticles or nanoparticles,
iii) mixing the microparticles or nanoparticles of step (ii) optionally with viscosity enhancing agent(s) and/or optionally with one or more excipients to form component-1,
iv) mixing the water miscible solvent optionally with viscosity enhancing agent(s) and/or other excipients to form component-2, and
v) mixing the component-1 and component-2 to obtain the desired composition before administration.

63. A process for the preparation of injectable composition according to claim 37, which comprises of the following steps:
i) dissolving the active agent(s) and biodegradable polymer(s) in an appropriate solvent and spray drying to form microparticles or nanoparticles
ii) mixing the microparticles or nanoparticles of step (i) optionally with viscosity enhancing agent(s) to form component-1,
iii) mixing the water miscible solvent optionally with viscosity enhancing agent(s) and/or other excipients to form component-2, and
iv) mixing the component-1 and component-2 to obtain a suitable injectable dosage form before administration.

64. A method of forming a depot gel or an implant in situ, in a living body, which comprises preparing an in situ gelling formulation according to claim 37, placing the formulation within the body and allowing the liquid vehicle to disperse or dissipate to produce a solid or gel implant.

65. A pharmaceutical kit suitable for in situ formation of a biodegradable depot gel or implant from the novel compositions according to claim 37, in the body of a subject in need thereof, which comprises a device containing tamsulosin or letrozole microparticles and optionally one or more pharmaceutical acceptable excipient(s), and a device containing liquid vehicle and optionally one or more pharmaceutical acceptable excipient(s); wherein the devices allow for expulsion of the contents of the two devices for enabling mixing together prior to administration of the contents into the body of the subject.

66. A method for the treatment of a condition treatable by tamsulosin or letrozole in a mammal particularly a human being by use of an in situ gelling formulation or an implant composition according to claim 37.

67. A method of using the compositions of tamsulosin or letrozole according to claim 37 which comprises administering to a subject/patient in need thereof an effective amount of the said composition.

68. A method of prophylaxis, amelioration and/or treatment of signs and symptoms of benign prostatic hyperplasia which comprises administering to a subject/patient in need thereof an effective amount of the composition according to claim 37 comprising tamsulosin as the active agent.

69. A method of prophylaxis, amelioration and/or treatment of hormonally-responsive breast cancer which comprises administering to a subject/patient in need thereof an effective amount of the composition according to claim 37 comprising letrozole as the active agent.

70. A method of using the compositions according to claim 37 for the treatment of benign prostatic hyperplasia comprising tamsulosin as the active agent

71. A method of using the compositions according to claim 37 for the treatment of hormonally-responsive breast cancer comprising letrozole as the active agent.
